(19) United States
(12) Patent Application Publication
(10) Pub. No.: US 2013/0064903 A1
(43) Pub. Date: Mar. 14, 2013

(54) RHEUMATIOD ARTHRITIS CURE

(71) Applicant: Lucille Townsend-Byrd, Cleveland, TN (US)

(72) Inventor: Lucille Townsend-Byrd, Cleveland, TN (US)

(21) Appl. No.: 13/604,424

(22) Filed: Sep. 5, 2012

Related U.S. Application Data
(60) Provisional application No. 61/573,546, filed on Sep. 8, 2011.

(51) Int. Cl. A61K 33/06; A61P 29/00; A61P 19/02

(52) U.S. Cl. 424/682

(57) ABSTRACT

A cause and cure for Rheumatiod Arthritis which includes the body becoming acid and retaining fluids as a means of diluting said acid. Calcium with adequate vitamin D for absorption neutralizes said acid and brings the body back in balance. In addition, an overabundance of the mineral potassium slows the removal of fluid from the body causing fluid to be retained and stored in the joints and soft tissues. Said storage of retained fluid is what causes the symptoms of rheumatoid arthritis and related illnesses and even fibromyalgia and headaches. Joints become swollen and prolonged inflammation results in joint deformity. Said abundance of potassium also causes an imbalance of other minerals in the body, especially sodium, resulting in dehydration. Water and balanced electrolytes must be added to correct the problem.

The root cause of Rheumatoid Arthritis is a sensitivity to the mineral potassium. For years I have read and heard that the cause and cure for rheumatoid arthritis were not known and that scientists were not even looking for a cure. When I was diagnosed with the disease 17 years ago it became necessary for me to know what was causing such searing pain and swelling in all of my joints. I have come from being bedridden and crippled having swollen fingers, hands, knees, ankles, and elbows that I was unable to hold a fork to feed myself and now without the use of any medication I can write with a pen.

Although potassium can be an important mineral, in some people the mineral acts as a drain stopper causing the body to retain fluids. Any liquids that are ingested remain in the body instead of leaving the body through normal elimination. Although the body will eliminate some urine, it will be slow and only a small amount. Much like a slow drain in a sink any additional liquids will force the body to find other places to store the overflow until it gradually recedes. An ultrasound will show normal kidneys but their function is hampered by the mineral potassium. It is like waiting overnight for a pint of water to go down the kitchen sink. The excess fluids take up residence anywhere and everywhere. This is why the joints of RA patients are deformed. The knuckles and other joints swell as a result of excess fluid around joint causing them to expand and become twisted and deformed. The longer the inflammation remains around the joint the more deformed the bone becomes. It is the inflammation that causes the pain and the deformity. Mystery pains in the arms, shoulders, and back called fibromyalgia are just the result of excess fluids not having anywhere else to go. Even some headaches are the pressure of an overflow of fluid in the brain. The body has nowhere to store it so the body swells inside and out until the kidneys can slowly drain it out.

Also, the build-up of acid in the body must be neutralized with calcium and vitamin D for calcium absorption or the body will retain fluids in an attempt to dilute said acid. It should be noted that a greater amount of vitamin D than the recommended amount is needed.

What can be done?

1) Dialysis comes to mind but doesn't seem feasible for the millions of RA patients. It also doesn't cure the disease.

2) Cutting back on fluid intake. I have also discovered that what you drink also makes a difference. Every fruit juice that I can find naturally has potassium in it. So cutting back on fruit juices will not help. As long as the drink has potassium it will cause inflammation, especially if a large amount is consumed. Drinking water, coffee, beer, or even sodas not flavored with fruit juices is what relieves the symptoms of RA. But even these beverages should be taken in moderation because they also disrupt the delicate potassium-sodium balance although not as much as the beverages with potassium. Some people are not willing to part with their morning orange juice or the healthy carrot, grape, or apple juices. And everyone is now drinking the exotic acai, noni, and pomegranate juices. These may contain antioxidants but they contain potassium and vitamin C. They are a disaster for RA patients. The potassium and vitamin C in the drinks will cause the kidneys to just about shut down. Vitamin C is almost as bad as the potassium.

It should be noted that although the mineral magnesium does not cause the inflammation, it exacerbates the problem because magnesium increases circulation so that the fluid in the inflammation is moved around the body to different joints and to the muscles sometimes settling in the ankles and knees making it impossible to walk or settling in the soft tissue of the arms making it impossible to raise the arms to brush the teeth or comb the hair.

Rheumatoid Arthritis is not an autoimmune disease where the body's own immune system is attacking itself. RA is a hypersensitivity to potassium and vitamin C. The disease modifying drugs that are available and which I have tried over the years don't work. They modify the disease, they don't cure it. It is the effects of potassium and vitamin C on the body that must be modified.

A major United States pharmaceutical company is interested in partnering with me to conduct a study to prove my findings and do more research on the effects of potassium and vitamin C on the body and how to modify such effects.

Please recognize that time is of the essence because RA is not a silent or pain free disease. It is in fact one of the most painful, if not the most painful disease. Millions are in agony every minute of everyday with no explanation of why. It is like being on fire without being consumed. I believe that like the disease polio, RA will be a disease of the past.

It is said that to defeat an enemy you must first understand it. I understand rheumatoid arthritis and have defeated it. Further study will prove my findings to be true for the general public. The patent is to protect my proprietary interests.

What is claimed is:

1. A discovery of the cause of and cure for rheumatoid arthritis and related illnesses, comprising of:
(a) a finding that the body in an acidic state attempts to neutralize acidity by retaining fluids and storing it in the joints, head, and soft tissues causing inflammation, pain and joint deformity . . . the symptoms of rheumatoid arthritis, fibromyalgia, headaches, and even brain fog and tiredness, said inflammation is what causes the pain of rheumatoid arthritis and long term swelling around the joints is the cause of joint deformity;
(b) the mineral calcium, as the body's neutralizing agent, with enough vitamin D for calcium absorption, eliminates the symptoms of rheumatoid arthritis and these related illnesses characterized by inflammation by extinguishing the flames of inflammation and pain as a fire extinguisher; acid is neutralized, proper ph is restored, and the body is brought back into balance; there is no more need for the body to hold fluids, said inflammation gradually decreases, the pain is gone, and the dis-ease is cured.

2. An observation that kidney function is affected by the mineral potassium and vitamin C and is directly related to the inflammation, pain, joint deformity, and tiredness characteristic of rheumatoid arthritis and related illnesses; said kidney disfunction results in fluid retention and since said fluid has no where to go it is stored in various parts of the body being the source of muscle pains, joint pain from fluids settling in the joints, swelling and deformity from water pressure around the joint, and headaches from excess fluid circulating throughout the body and head;
(a) the abundance of said potassium also results in dehydration from an imbalance with other electrolytes, especially sodium, further exacerbating the problem . . . more pain and swelling; if more fluids are ingested in an attempt to relieve dehydration, the body stores it and the inflammation becomes worse because of the effects of acidity as claimed in claim 1 and the effects of potassium and vitamin C;
(b) the effects of vitamin C on the kidneys is also a problem for rheumatoid arthritis sufferers, said vitamin C hampers kidney function and contributes to fluid retention.
(c) the circulatory function of the mineral magnesium, although not a primary cause of the pain and swelling of these diseases, moves said excess fluid buildup throughout the body where said fluid settles especially in the extremeties, (ie.), the ankles and knees.

3. A useful pharmaceutical for modifying the effects of potassium and vitamin C and neutralizes acid in the body requiring large scale studies will be developed and marketed to the public.
